(19) United States
(12) Patent Application Publication
(10) Pub. No.: US 2007/0172507 A1
(43) Pub. Date: Jul. 26, 2007

(54) Transluminal drug delivery methods and devices

(76) Inventors: Paul Zupkas, San Diego, CA (US); Charles Lowell Parsons, San Diego, CA (US)

Correspondence Address: CATALYST LAW GROUP, APC, 9710 SCRANTON ROAD, SUITE S-170, SAN DIEGO, CA, 92121, US

(21) Appl. No.: 11/340,071

(22) Filed: Jan. 26, 2006

(51) Int. Cl. A61K 31/737; A61K 31/728; A61K 31/727; A61K 31/24

(52) U.S. Cl. 424/423; 514/54; 514/56; 514/536

(57) ABSTRACT

Transluminal drug delivery method and device embodiments may include a urethral suppository formulated to treat diseases of the urethra and surrounding organs by enhancing the absorption of a therapeutic agent of the suppository into body tissues without adversely affecting the natural defense mechanisms of these tissues. Adverse affects on the glucosaminoglycan (GAG) barrier by the suppository may be mitigated or eliminated by the presence of a suitable polysaccharide in the suppository.

### BACKGROUND

Diseases of the urinary tract present a growing healthcare problem worldwide. One of the most common diseases of the urinary tract is interstitial cystitis. Interstitial cystitis (IC) is a clinical syndrome of frequency, urgency, and/or pelvic pain in the absence of any other definable pathology, such as urinary infection, carcinoma, or cystitis induced by radiation or medication. A diagnosis of IC is often reached as a diagnosis of exclusion, where patients have tried and failed treatments for other diseases exhibiting similar symptoms. The disease is best understood as a continuum, with an early phase in which symptoms are intermittent, a middle phase in which symptoms may be chronic and flare episodically, and sometimes a late phase in which bladder destruction occurs. In its early stages, IC is often mistaken for other urologic or gynecologic disorders, and tends to go unrecognized until its advanced stages. Treatment options for IC are limited. There are few oral or intravesical medications that have shown efficacy in treating IC. Physical intervention in the form of a cystectomy is used as a last resort in end stage disease.

Although oral medications, such as pentosanpolysulfate and hylauronic acid, are used to treat IC by replacing missing components in a defective glucosaminoglycan (GAG) barrier, a fundamental characteristic of IC, the use of oral anesthetic agents to inhibit urinary tract sensory nerve activation is impractical. Oral drug delivery affects the entire body requiring high oral doses of drugs to achieve therapeutically significant levels in a target organ. When the organ is in the urinary tract, such as the bladder or urethra, oral drugs must pass through and be affected by other organs before reaching their target. This effect may change the activity or function of the drug resulting in undesirable side effects or other co-morbidities. An alternative to oral delivery is topical or site specific delivery of drug, where the drug is delivered directly to the diseased organ. Topical drug delivery generally provides similar efficacy at lower drug doses than oral and may reduce or eliminate the effect of the drug on any other than the target organ.

The use of urethral suppositories for topical drug deliver has been known, however, existing modalities do not allow for an efficient absorption of some beneficial therapeutic agents. What has been needed are systems and methods for efficient delivery of therapeutic agents to at least a portion of the tissue of a patient's urinary tract, or surrounding tissue thereof.

### SUMMARY

Some embodiments of a urethral suppository include a carrier base material, an anesthetic agent and a buffering agent formed into a solid structure configured for insertion into a patient's urethra. Such embodiments may include a a polysaccharide and a substantially uniform composition.

Some embodiments of a method for manufacturing a urethral suppository include combining an anesthetic agent and a buffering agent in a liquid carrier base material until the anesthetic agent and buffering agent have dissolved or suspended in the liquid carrier base material. The liquid carrier base material, anesthetic agent and buffering agent mixture are then formed into a suppository that is configured to be deployed within a patient's urethra. Such embodiments may also include combining a polysaccharide with the liquid carrier base material prior to the formation of a suppository.

Some embodiments of a method of treating at least a portion of a patient's urinary tract include providing a urethral suppository having an anesthetic agent, a buffering agent and a carrier base material. The urethral suppository is deployed within the patient's urethra and thereafter the suppository is allowed to at least partially disintegrate and release the anesthetic agent and buffering agent.

Some embodiments of a depot for luminal drug delivery include a carrier base material, an anesthetic agent and a buffering agent formed into a solid structure configured for insertion into a body lumen of a patient. Such embodiments may also include a polysaccharide.

These features of embodiments will become more apparent from the following detailed description when taken in conjunction with the accompanying exemplary drawings.

### BRIEF DESCRIPTION OF THE DRAWINGS

FIG. 1 is a perspective view of an embodiment of a combination of a carrier base material, an anesthetic agent, a buffering agent and a polysaccharide in liquid form being poured into a suppository mold cavity embodiment.

FIG. 2 is a perspective view of an embodiment of a combination of a carrier base material, an anesthetic agent, a buffering agent and a polysaccharide in liquid form being poured into a suppository mold cavity embodiment.

FIG. 3 is a perspective view of a urethral suppository embodiment.

FIG. 4 is a side view in partial section of a distal portion of a delivery catheter disposed within a urethra of a patient and a urethral suppository being advanced distally within the delivery catheter.

FIG. 5 is a side view in partial section of the urethral suppository disposed within the urinary tract of the patient with the delivery catheter withdrawn.

### DETAILED DESCRIPTION

There is currently no definitive diagnostic test for IC, although tests such as the Potassium Sensitivity Test have been used to identify defects that are fundamental characteristics of IC. The Potassium Sensitivity Test (PST) uses a pain, urgency, and frequency questionnaire to identify patients who exhibit a negative reaction to the injection of a concentrated potassium solution in their bladder. The basis for the test is the identification of a fundamental characteristic of IC, which is a breakdown of GAG barrier. Breakdown of the GAG barrier allows components in urine, such as potassium, to move into the interstitial spaces. As these components move into the interstitial spaces, they activate sensory nerves that result in pain and begin an inflammatory response. The inflammatory response begins a cascade of events, including the activation of mast cell mediators, further activating sensory nerves resulting in more intense and prolonged pain. The inflammatory response is also considered to play a key role in the breakdown of the GAG layer. A objective in the treatment of IC is to reduce the inflammatory response by inhibiting the activation of sensory nerves. Inhibiting nerve activation reduces the patient's pain and slows or stops the degradation of the GAG barrier. Inhibition of sensory nerves can be achieved using anesthetic drugs, such as lidocaine and procaine, as well as others, which serve to inhibit ionic fluxes required for the initiation and conduction of nerve impulses.

In the urinary tract, the topical delivery of drugs requires overcoming the natural GAG barrier that prevents materials from moving from the luminal space into the interstitium of the urinary tract. Thus, for a topically delivered drug to effectively treat IC, it must pass through the GAG barrier to reach urinary tract tissues. At the same time, as it passes through the barrier, the drug must not disable or damage the barrier or negatively effect its function to block harmful components in urine. Embodiments discussed herein include method for effectively delivering topical drugs or therapeutic agents to urethral tissue, surrounding tissue or both, without adversely affecting the GAG barrier of the urinary tract.

Although IC is considered a disease of the bladder, researchers have described a urethral component to the disease. This is not surprising as many neural and systemic networks are shared by the bladder and urethra. Unlike the bladder, the urethra is a collapsed tube in its resting state and opens to allow urine pass out of the bladder. Therefore, any liquid or gel material placed in the urethra would be pushed out of the urethra into the bladder or out of the body. To treat the urethra, a medication may be incorporated into a structure that will be retained in the urethra for periods of minutes to hours. Embodiments of a drug delivery system includes a suppository base as a means to expose the urethra to medication for periods of time from minutes to hours. The formulation of the delivery system including the type of base materials used as a delivery vehicle, the concentration of drug, and the ratio of drug to buffering agent may be chosen so as to produce an efficient mechanism for delivering the therapeutic agent. Size may be an important aspect for the performance and patient tolerance of a urethral suppository and is a consideration in any suppository formulation. The female urethral is approximately 3-4 cm in length. Most patients can tolerate object placed in their urethras up to about 19 Fr (6.3 mm), without major discomfort. The maximum size of a urethral suppository that comfortably fits in the female urethra is about 2.5 cm in length and about 0.65 cm in diameter or transverse dimension. One difficulty with the performance of previously available urethral suppositories has been formulating a suppository that fits comfortably in the urethra yet contains enough drug to produce the desired therapeutic effect while maintaining the functional characteristics of a suppository.

Embodiments include a urethral suppository formulated to treat diseases of the urethra and surrounding organs by enhancing the absorption of drug into body tissues without adversely affecting the natural defense mechanisms of these tissues. This unique formulation is adapted to allow the active therapeutic agent to pass through the GAG barrier that lines the urethra and the urinary tract. Adverse affects on the GAG barrier by the suppository may be mitigated or eliminated by the presence of a suitable polysaccharide formulated in the suppository.

Embodiments of urethral suppositories include the use of a therapeutic agent which is an anesthetic agent in a suppository mixture. Anesthetic agents, such as lidocaine, a neuronal sodium channel blocking agent, may be used as well as other types of agents. Lidocaine is a white solid substance typically provided as lidocaine hydrochloride. When mixed with water, this form of lidocaine creates an ionic solution that is acidic. For 2-5% lidocaine solutions in sterile water, the pH range is 5-6. It is known; the ionic form of lidocaine is not readily absorbed into tissues of many body cavities and organs. The mechanism for this phenomenon is unclear. However, buffering ionic lidocaine converts it to a lipid soluble form of the drug. This form of the drug is more readily absorbed into tissues. We believe that buffering effects the charge of the lidocaine molecule making it more non-polar. As a non-polar species, the lidocaine passes through the GAG barrier lining all organs in the urinary tract that blocks the ionic form of the drug.

Lidocaine is a fairly non-polar molecule by itself. When mixed with water, the presence of an amide bond and a secondary amine group make the free lidocaine molecule a weak base. Generally a weak base will bond protons lightly when the protons are present in excess. Because lidocaine exists as a weak base in its uncharged form, the ionized species of lidocaine hydrochloride will predominate when mixed in solutions whose pHs are lower than the pKa of lidocaine, or when there are suitably high concentrations of H+. When the pH of the solution is equal to the pKa of lidocaine the ionized and non-ionized forms of lidocaine will be present in equal amounts. When the pH of the solution, such as water, is less than that of lidocaine, there are enough free protons in solution for lidocaine to bond to one proton and acquire a positive charge. This charge hinders the lidocaine absorption across any type of mucous layer. At a pH greater than its pKa, the non-ionized or free form of the molecule predominates, as there are no protons for lidocaine to abstract. The pKa of ionized lidocaine is 7.8. Writing out the Henderson-Hasselbach relationship and reducing the equation in terms of pH and pKa gives;

$$\frac{[ NonIonized ]}{[ Ionized ]} = {10}^{pH - pKa} .$$

Lipid molecules are a primary component of cell membranes and are known to be extremely hydrophobic. In the bladder and urethra, the lipid membranes of the urothelial cells are buffered from direct contact with urine by the presence of the GAG barrier. GAG molecules readily attract water molecules creating a hydrolyzed molecule that is a primary component of the layer that lines the urethra and bladder luminal wall. The water molecules in the mucous layer have a slight negative charge that repels other negatively charged ionic species. In alkali buffered solutions, defined as a solution whose pH is greater than 7.0, lidocaine will exist predominantly in its uncharged form. The ratio of uncharged lidocaine to charged lidocaine increases significantly as the pH of the solution rises above 7.8. Since non-polar molecules are more lipophillic than hydrophilic, they easily and efficiently pass through the mucous layer and cell membranes of the bladder and urethral tissue cells.

Several carrier base materials and buffering agents may be used to create a buffered lidocaine urethral drug delivery system, however, some materials and agents may perform better than others. Embodiments of carrier base materials may include gelatins, polyethylene glycols (PEGs), glycerols, methyl paraben, propyl paraben, hydrated vegetable oil and cocoa butter, methyl butyl ketone (MBK), other fatty acid bases and the like. Embodiments of buffering agents may include sodium bicarbonate, sodium hydroxide, calcium bicarbonate, magnesium oxide, potassium hydroxide, sodium carbonate, tris(hydroxylmethyl)aminomethane and the like. One useful carrier base material and buffering agent combination includes MBK and sodium bicarbonate. The melting point of MBK may be adjusted to alter it's disintegration rate via a melting process over a wide range of time at body temperature. Adjustment of the melting point of MBK may be carried out by adding selected amounts of paraffin. MBK also has an affinity to dissolve lidocaine. Sodium bicarbonate may be useful as a buffering agent for some embodiments of urethral suppositories because it has the ability to hold a pH in the range of 8-9 in combination with lidocaine and the carrier base. In order to obtain data on suppository embodiments, some testing was performed using some or all of the components discussed above.

### EXAMPLE 1

Suppository Fabrication

Animal tests, using a New Zealand White Rabbits model, were conducted to determine the safety profile of buffered lidocaine absorption by measuring plasma levels of lidocaine in the systemic circulation using different carrier bases. Levels of lidocaine that would be toxic if injected intravenously, were fabricated in a suppository carrier base. Different carrier bases were warmed in a 40° C. bath and lidocaine was added in increments of 10% of the final weight of the mixture until it reached a ratio of 3:7 lidocaine to carrier base. The pH of the mixture was measured after all lidocaine had dissolved using a temperature compensating pH meter. 30% lidocaine mixture was found to be the highest concentration lidocaine mixture achievable with the carrier base materials that were tried. The mixture was raised to 70° C. in a water bath and titrated with sodium bicarbonate to a pH of 8.5 while gently stirring. Heating and stirring was stopped when the mixture lost its grainy appearance and became clear as all components dissolved in the carrier base. The mixture was drawn into tuberculin syringes, refrigerated overnight to form a suppository, and extruded the next day. The extruded suppository was cut into thirds forming elongate sections having a length of about 2 cm and containing approximately 100 mg of lidocaine.

Urethral Insertion

Female NZW Rabbit subjects having a weight range of about 3.5 kg to about 3.8 kg were anesthetized using IM injections of Ketamine and Xylazine followed by half doses very hour as needed. The common carotid artery was exposed and cannulated to provide blood pressure measurements using a Life-Tech BP2110 pressure transducer and to draw blood. Another incision was made in the abdomen to expose the bladder. Through a small incision in the dome of the bladder, a 7 Fr pediatric feeding tube was placed in the bladder. All urine was drained from the bladder. The tube was fed out the bladder through the urethra and out the urethral opening. The suppositories were attached to lengths of 3-0 silk sutures that were fed through the tube and out the bladder incision. The tube was removed and the suture pulled through the incision, drawing the suppository into the urethra. The bladder incision was closed. Blood samples were drawn at 0, 15, 30, 60, and 90 minutes. After 90 minutes, the animals were sacrificed and their urethras removed and placed in a 10% formaldehyde solution for histological study.

Results

Free lidocaine was measured in plasma samples using the Abbot Laboratories TDx sheep albumin immunoflourescence assay. The assay is accurate for lidocaine levels from about 1-7 μg per ml of plasma. This assay was chosen for the reason that it is the standard means by which lidocaine concentration is measured for therapeutic purposes or in cases of suspected toxicity. In all carrier base mixtures, the maximum concentration of lidocaine in the blood was less than the resolution of the assay at 60 and 90 minutes after introduction. The maximum concentrations were observed between 15 and 30 minutes and did not exceed 1.1 μg of lidocaine per ml of plasma in all subjects. No subject had a measurable amount of lidocaine in its blood after 30 minutes. No significant hemodynamic changes were observed. The level of lidocaine administered to all subjects was a full order of magnitude greater than the currently prescribed clinical dose of analgesic lidocaine. Even at this elevated dose, the blood levels of lidocaine did not reach the threshold for a therapeutic effect (2-5 μg per ml) or toxic level (greater than 8 μg per ml). Histological examination of post-insertion urethras using H&E stain revealed an inflammatory response in epithelial cells lining the luminal urethra in suppositories using a PEG carrier base. All other suppository carrier materials revealed normal cellular structure in the urethral cells.

No subject voided after insertion of the lidocaine urethral drug delivery mixture or after instillation of an alkanized lidocaine solution. These results suggest that a buffered urethral delivery of lidocaine reduces patient exposure to toxic lidocaine levels, as evidenced by the low peak values of plasma lidocaine. In addition the cessation of voiding after the administration of lidocaine imply that the drug is acting as a local analgesic in the bladder and urethra. In such a case the amount of lidocaine transported into the tissue was sufficiently great to block nerve conduction and provide analgesia without exposing the subject to perilous plasma lidocaine concentrations.

### EXAMPLE 2

Human clinical studies were conducted to determine the efficacy of a buffered lidocaine suppository having a mucopolysaccride component to repair any defect or injury to a luminal surface of a patient's urethra. A conical suppository with a volume of approximately 500 mg was used in the study. Suppositories were fabricated to contain approximately 10% lidocaine buffered to a pH of about 7.8. Methyl butyl ketone (MBK) was chosen as the suppository carrier base material, because the melting time of the base material could be adjusted by addition of paraffin to a range of 5-15 minutes. Suppositories were fabricated with PEG but without any active ingredient to determine if the inflammatory response observed in the animals resulted in any adverse effects in humans. Suppositories containing PEG carrier base materials were placed in two human subjects with both subjects complaining of urethral burning and urinary frequency. As a result, PEG was not used clinically in any further suppository formulation in the study. The suppository formulation used in the clinical study was as follows:

Therapeutic Agent

| | | |
|---|---|---|
| Lidocaine | 45 | mg |
| Heparin | 5000 | units |
| Sodium Bicarbonate | 10 | mg |
| Silica | 10 | mg |

Base Material

| | | |
|---|---|---|
| MBK | 383 | mg |
| Paraffin | 67 | mg |

These carrier base materials were melted in a water bath at 60° C. and thoroughly mixed. The lidocaine, heparin, sodium bicarbonate, and silica were added to the carrier base materials while gently stirring the mixture. After all suppository components were dissolved, the mixture was drawn into a syringe and injected in the cavities of the suppository mold. The mold was refrigerated overnight, after which the suppositories are removed from the mold and individually packaged. All suppositories were stored at 5° C. prior to use.

A total of 25 patients with a clinical diagnosis of IC with a urethral component determined by a score of greater than 15 out of 20 on a pain, urgency, and frequency (PUF) questionnaire received the buffered lidocaine suppositories. Patients were asked to grade their level of pain and discomfort pre and post insertion of the suppository. Before insertion of the suppository, patients graded their pain and discomfort using a questionnaire at 8 on a 10 point scale with 10 being the highest degree of pain and discomfort. Patients graded their pain and discomfort using the same questionnaire 30 minutes after insertion of the suppository at 3 on a 10 point scale.

Embodiments of urethral suppositories may be formed by mixing or combining components into a mixture and forming the mixture into a solid suppository. Embodiments of methods for manufacturing a urethral suppository may include combining a therapeutic agent in the form of an anesthetic agent and a buffering agent in a liquid carrier base material. For some embodiments, a polysaccharide may also be combined with the liquid carrier base material before formation of the suppository. The combined components may be stirred, heated or both until the anesthetic agent and buffering agent have been dissolved or suspended in the liquid carrier base material. For example, the carrier base material may be warmed in a bath having a temperature of about 35 degrees C. to about 45 degrees C. Once the carrier base material is warmed, an anesthetic agent, such as lidocaine, may be added. After the anesthetic agent has dissolved or been otherwise suspended in solution, the mixture may continue to be heated or have the temperature raised to a temperature of about 60 degrees C. to about 80 degrees C. Thereafter, the mixture may be titrated with a buffering agent, such as sodium bicarbonate, to a pH of about 7.5 to about 9 while gently stirring. For some embodiments, the mixture may lose its grainy appearance and became clear as all components dissolved in the carrier base material. Once the components have been suitably mixed, the liquid carrier base material, anesthetic agent and buffering agent mixture may be formed into a suppository or other type of solid depot that is configured to be deployed within a patient's urethra or other body lumen.

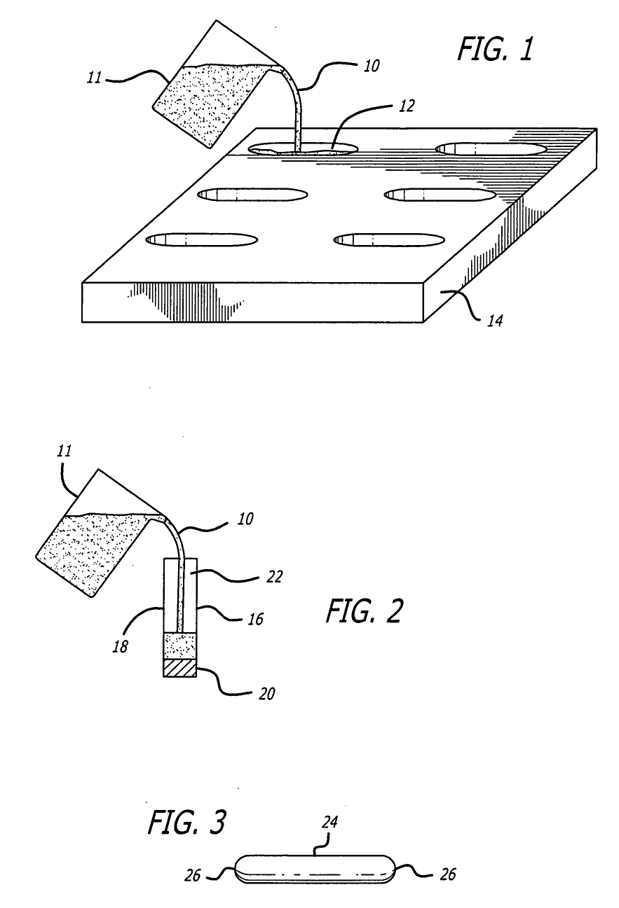

FIG. 1 shows a perspective view of an embodiment of a combination of a carrier base material, an anesthetic agent, a buffering agent and a polysaccharide in a mixture 10 in liquid form being poured from a container 11 into a suppository mold cavity 12. A mold body 14 includes 6 individual mold cavities 12 and each individual mold cavity 12 has an elongate configuration with rounded or spherically shaped ends. Once one or more of the mold cavities 12 have been filled with the combined mixture 10, the combined mixture 10 is allowed to harden and form a solid structure. In some cases, the mold body 14 may be refrigerated overnight, after which the mixture 12 which has hardened into suppositories are removed from the mold 12 and individually packaged. The suppositories may also be stored at lowered temperatures, such as about 0 degrees C. to about 10 degrees C. prior to use.

FIG. 2 is a perspective view of mixture 10 being poured from container 11 into a cylindrical suppository mold embodiment 16. The suppository mold 16 has a barrel shaped body portion 18 having a substantially round cross section with a removable plug 20 disposed at the bottom of a mold cavity chamber 22. After the mold cavity chamber 22 has been filled to a desired level, the combined carrier base material, anesthetic agent, buffering agent and polysaccharide mixture 10 is allowed to harden. Thereafter, the plug 20 may be removed and the newly formed suppository pushed from the mold cavity 22. The plug 20 may also be used to push the newly formed suppository from the mold cavity 22.

FIG. 3 is a perspective view of a urethral suppository embodiment 24. The suppository 24 has an elongate cylindrical configuration with rounded or spherically shaped ends 26, however, other configurations such as conical or ellipsoidal may also be used. As discussed above, the size of a suppository 24 may be an important aspect for some embodiments. In particular, for urethral suppositories 24, it may be useful to have as large a suppository 24 as possible while maintaining an acceptable level of patient discomfort. Some embodiments of a urethral suppository 24 may have a length of about 5 mm to about 50 mm, specifically, about 15 mm to about 35 mm. Such embodiments may have a transverse dimension or diameter of about 1 mm to about 10 mm, more specifically, about 3 mm to about 6 mm. The overall weight of some embodiments of the suppository 24 may be about 10 mg to about 1000 mg, specifically, about 400 mg to about 600 mg.

The carrier base material for the suppository 24 may include a variety of suitable materials. Materials such as paraffin, theobroma oil, modified theobroma oil products, glycerinated gelatin, hydrogenated vegetable oils, paraben base products, methyl butyl ketone (MBK), cellulose, poly(vinyl alcohol), poly(vinylpyrrolidone), polyacrylamide, poly(phospho urethanes), polyoxyl stearate, ethylenoxide polymers and the like may be used individually or in combination. A carrier base material or materials having a melting point of about 36 degrees C. to about 38 degrees C. may be useful. In addition, a carrier base material that is water soluble may also be useful. The melting time of embodiments of the base material may be adjusted by the addition of paraffin to the liquid mixture to achieve a melting time of about 5 minutes to about 15 minutes.

Suitable anesthetic agents for the suppository 24 may include lidocaine, a neuronal sodium channel blocking agent, or other agents such as prilocaine, benzocaine, mepivicaine, etidocaine, articaine, bupivicaine, procaine and tetracaine. The anesthetic effect of all of these agents will increase when mixed with suitable buffers. However, by mixing buffering agents at different concentrations with these anesthetic agents, different anesthetic effects over varying periods of time may be achieved. It may be useful for some embodiments to have therapeutic agent that is configured to inhibit the conduction or initiation of nerve impulses. Some embodiments of the urethral suppository 24 may include about 1 mg to about 100 mg of anesthetic agent, specifically, about 30 mg to about 60 mg of anesthetic agent.

Buffering agents that may be used for embodiments of the suppository 24 may include buffering agents configured to maintain a pH of the urethral suppository of about 7 to about 12, specifically, about 7.5 to about 9. As discussed above, embodiments of buffering agents may include sodium bicarbonate, sodium hydroxide, calcium bicarbonate, magnesium oxide, potassium hydroxide, sodium carbonate, tris(hydroxylmethyl)aminomethane and the like. Combinations of different buffering and anesthetic agents will have different stability properties, affecting their efficacy when stored for long periods of time. The choice of buffering agent or agents may also be driven by the ability of a buffering agent to maintain a uniform distribution of the anesthetic agent throughout the suppository 24. Some embodiments of the urethral suppository 24 may include about 0.5 mg to about 100 mg of buffering agent, specifically, about 1 mg to about 20 mg of buffering agent. Some embodiments of urethral suppositories 24 may contain a buffering agent or agents of about 1 percent to about 20 percent by weight of the overall weight of the suppository 24. Some embodiments of urethral suppositories 24 may include a buffering agent or agents of about 5 percent to about 10 percent by weight with respect to the weight of the anesthetic agent of the suppository 24.

The composition of some embodiments, and particularly, some of the buffering agents, may include pharmaceutically acceptable salts. Pharmaceutically acceptable salts may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate(embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20.sup.th ed.) Lippincott, Williams & Wilkins (2000) which is incorporated herein in its entirety. Additional pharmaceutically acceptable salts may include, for example, acetate, benzoate, bromide, carbonate, citrate, gluconate, hydrobromide, hydrochloride, maleate, mesylate, napsylate, pamoate(embonate), phosphate, salicylate, succinate, sulfate, or tartrate.

Embodiments of the suppository 24 may include a polysaccharide material, such as mucopolysaccharide, configured to replace or repair the GAG layer or barrier of at least a portion of a patient's urinary tract, such as the urethra. Polysaccharides that may be used for various embodiments of urethral suppositories 24 include pentosanpolysulfate, heparin, hylaronic acid, heparan sulfate, oligosaccharide, chondroitin sulfate, keratin sulfate or other glycosaminoglycans or polysaccaride. Some embodiments of the urethral suppository 24 may include about 0.5 mg to about 100 mg of polysaccharide, specifically, about 1 mg to about 20 mg of polysaccharide. Silica may also be added to the mixture 12 and suppository 24 as a suspending agent to prevent active ingredients in the mixture 12 of the suppository 24 from aggregating. A percentage by weight of silica that may be useful for some embodiments of a suppository 24 may include about 0.1 percent to about 5 percent by weight of the suppository 24.

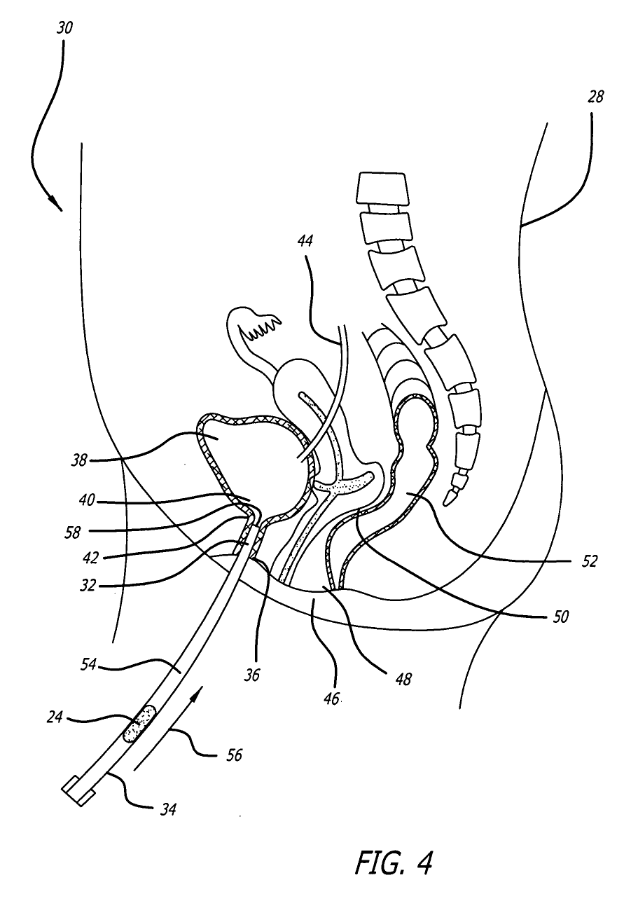

Once the urethral suppository 24 has been formed or otherwise manufactured, it may be deployed within the body 28 of a patient 30 in order to treat a variety of conditions including pain, IC, pre-procedure desensitization as well as others. Although a human patient is depicted in FIG. 4, and the embodiments discussed herein would typically be used for human patients, they may also be used in veterinary medicine to treat similar or identical indications in animals. FIG. 4 is a side view in partial section of a distal portion 32 of a delivery catheter 34 disposed within a urethra 36 of a patient. FIG. 4 illustrates the patient's urinary tract including the bladder 38, bladder neck 40, urethra 42 and ureter 44. Also shown are some of the tissue surrounding the patient's urinary tract including the labia minora 46, urethral os 48, vagina 50 and rectum 52. The urethral suppository 24 is shown being advanced distally within an inner lumen 54 of the delivery catheter 34 towards a urethra 36 of the patient 30, as indicated by arrow 56. The delivery catheter 34 is an elongate tubular member having a length of about 10 cm to about 100 cm, an outer transverse dimension or diameter of up to about 19 Fr. and an inner lumen having an inner transverse dimension or diameter of up to about 10 mm. The delivery catheter 34 may have any suitable construction included an extruded polymer tube that may optionally be reinforced with braided material or the like. Materials such as polyethylene, polyurethane, nylons or the like may be used. Once the suppository 24 has been advanced to the distal portion 32 of the delivery catheter 34 and ejected from a distal end 58 of the delivery catheter 34, the delivery catheter 34 may be withdrawn from the urethra 36 of the patient 30.

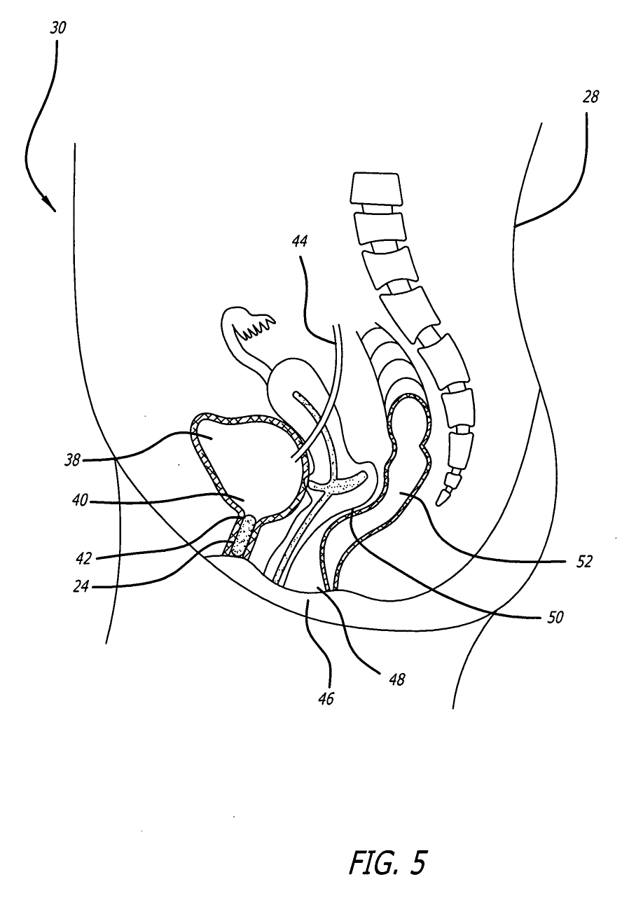

FIG. 5 shows the patient 30 of FIG. 4 with the suppository 24 disposed within the urethra 36 and the delivery catheter 34 withdrawn. Once the suppository 24 has been deployed within the urethra 36 or other body lumen, the carrier base material may begin to disintegrate and thereby deliver the buffered anesthetic or therapeutic agent to tissue of the patient's urinary tract and surrounding tissue. The disintegration of the suppository 24 may be carried out by dissolving of the carrier base material for some embodiments, particularly, embodiments having a water soluble carrier base material. The disintegration of the suppository 24 may also occur due to melting of the carrier base material as a result of exposure to body temperature within the urethra. In either modality, disintegration of the carrier base material exposes the therapeutic agent or agents integrated into the suppository structure 24.

The exact dosage delivered to patient 30 may depend on the subject to be treated, the body weight of the subject to be treated, and the preference and experience of the attending physician. The optimal concentration and dosage of the therapeutic agent to be delivered may also depend upon the specific anesthetic used, buffering agent used, carrier base material used and optional polysaccharide used, in addition to the characteristics of the patient, and the nature of the condition for which the treatment is sought. These factors can be determined by those of skill in the medical and pharmaceutical arts in view of the present disclosure. Generally, a therapeutically effective dose is desired. A therapeutically effective dose refers to that amount of the anesthetic agent that results in a degree of amelioration of symptoms relative to the status of such symptoms prior to treatment. The dosage forms containing effective amounts are within the bounds of routine experimentation and therefore, well within the scope of the embodiments discussed herein. In general however, a suitable dose of a buffered anesthetic for topical delivery may be in the range of from about 0.1 to about 10 mg/kg of body weight per day, specifically, in the range of about 0.2 to about 5 mg/kg/day, and more specifically, in the range of about 0.4 to about 2 mg/kg/day.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A urethral suppository comprising a carrier base material, an anesthetic agent and a buffering agent formed into a solid structure configured for insertion into a patient's urethra.

2. The urethral suppository of claim 1 further comprising a substantially uniform composition.

3. The urethral suppository of claim 1 further comprising a polysaccharide.

4. The urethral suppository of claim 1 wherein the carrier base material comprises a melting temperature of about 36 degrees C. to about 38 degrees C.

5. The urethral suppository of claim 1 wherein the carrier base material comprises a water soluble carrier base.

6. The urethral suppository of claim 1 wherein the carrier base material comprises MBK.

7. The urethral suppository of claim 1 wherein the carrier base material comprises at least one of the materials selected from the group consisting of theobroma oil, modified theobroma oil products, glycerinated gelatin, hydrogenated vegetable oils, paraben base products, methyl butyl ketone, cellulose, poly(vinyl alcohol), poly(vinylpyrrolidone), polyacrylamide, poly(phospho urethanes), polyoxyl stearate and ethylenoxide polymers.

8. The urethral suppository of claim 1 wherein the anesthetic drug is configured to inhibit the conduction or initiation of nerve impulses.

9. The urethral suppository of claim 1 wherein the anesthetic comprises at least one of the materials selected from the group consisting of lidocaine, prilocaine, benzocaine, mepivicaine, etidocaine, articaine, bupivicaine, procaine and tetracaine.

10. The urethral suppository of claim 1 wherein the buffering agent adjusts the pH of the urethral suppository to about 7 to about 12.

11. The urethral suppository of claim 1 wherein the buffering agent adjusts the pH of the urethral suppository to about 7.5 to about 9.

12. The urethral suppository of claim 2 wherein the polysaccharide comprises at least one material selected from the group consisting of pentosanpolysulfate, heparin, hylaronic acid, heparan sulfate, oligosaccharide, chondroitin sulfate, keratin sulfate or other glycosaminoglycans or polysaccaride.

13. The urethral suppository of claim 1 wherein the urethral suppository comprises a weight of about 10 mg to about 1000 mg.

14. The urethral suppository of claim 13 wherein the urethral suppository comprises a weight of about 400 mg to about 600 mg.

15. The urethral suppository of claim 1 wherein the urethral suppository comprises about 1 mg to about 100 mg of anesthetic agent.

16. The urethral suppository of claim 15 wherein the urethral suppository comprises about 30 mg to about 60 mg of anesthetic agent.

17. The urethral suppository of claim 1 wherein the anesthetic agent comprises lidocaine.

18. The urethral suppository of claim 1 wherein the urethral suppository comprises about 0.5 mg to about 100 mg of buffering agent.

19. The urethral suppository of claim 18 wherein the urethral suppository comprises about 1 mg to about 20 mg of buffering agent.

20. The urethral suppository of claim 2 wherein the urethral suppository comprises about 0.5 mg to about 100 mg of polysaccharide.

21. The urethral suppository of claim 20 wherein the urethral suppository comprises about 1 mg to about 20 mg of polysaccharide.

22. The urethral suppository of claim 1 comprising a shape selected from the group consisting of a cylinder, a cone and an ellipsoid.

23. The urethral suppository of claim 1 wherein the urethral suppository comprises an elongate structure with a transverse dimension of about 1 mm to about 10 mm.

24. The urethral suppository of claim 23 wherein the urethral suppository comprises an elongate structure with a transverse dimension of about 3 mm to about 6 mm.

25. The urethral suppository of claim 1 wherein the urethral suppository comprises an elongate structure with a length of about 5 mm to about 50 mm.

26. The urethral suppository of claim 25 wherein the urethral suppository comprises an elongate structure with a length of about 15 mm to about 35 mm.

27. The urethral suppository of claim 1 comprising a buffering agent having a weight of about 1 percent by weight to about 30 percent by weight of a weight of anesthetic agent combined with the liquid carrier base material.

28. A method for manufacturing a urethral suppository, comprising:
combining an anesthetic agent and a buffering agent in a liquid carrier base material until the anesthetic agent and buffering agent have dissolved or suspended in the liquid carrier base material;
forming the liquid carrier base material, anesthetic agent and buffering agent mixture into a suppository that is configured to be deployed within a patient's urethra.

29. The method of claim 28 further comprising combining a polysaccharide with the liquid carrier base material.

30. The method of claim 29 wherein combining a polysaccharide with the liquid carrier base material comprises combining at least one polysaccharide selected from the group consisting of pentosanpolysulfate, heparin, hylaronic acid, heparan sulfate, oligosaccharide, chondroitin sulfate, keratin sulfate or other glycosaminoglycans or polysaccaride.

31. The method of claim 28 wherein forming the mixture into a suppository is configured to produce a finished suppository having a weight of about 10 mg to about 1000 mg.

32. The method of claim 28 wherein combining the anesthetic agent comprises adding lidocaine.

33. The method of claim 28 wherein combining an anesthetic agent and a buffering agent in a liquid carrier base material comprises adding an amount of buffering agent sufficient to produce a pH of about 7 to about 12 in the finished suppository.

34. The method of claim 28 wherein combining an anesthetic agent and a buffering agent in a liquid carrier base material comprises adding buffering agent having a weight of about 1 percent by weight to about 30 percent by weight of a weight of anesthetic agent combined with the liquid carrier base material.

35. A method of treating at least a portion of a patient's urinary tract, comprising:
providing a urethral suppository including an anesthetic agent, a buffering agent and a carrier base material;
deploying the urethral suppository within the patient's urethra;
allowing said suppository to at least partially disintegrate and release the anesthetic agent and buffering agent.

36. The method of claim 35 wherein disintegration of the suppository comprises melting of the carrier base material.

37. The method of claim 35 wherein disintegration of the suppository comprises dissolving of the carrier base material.

38. The method of claim 35 wherein treating at least a portion of the patient's urinary tract comprises treatment of IC.

39. The method of claim 35 wherein the urethral suppository is deployed within the patient's urethra in order to desensitize the patient's urethra prior to insertion of instrumentation into the urethra.

40. The method of claim 35 wherein treating at least a portion of the patient's urinary tract comprises treatment of pain associated with the urethra or bladder.

41. The method of claim 35 wherein the urethral suppository further comprises polysaccharide and wherein the polysaccharide replaces or repairs the GAG barrier lining the urinary tract after insertion into the patient's urethra.

42. A depot for luminal drug delivery comprising a carrier base material, an anesthetic agent and a buffering agent formed into a solid structure configured for insertion into a body lumen of a patient.

43. The depot of claim 42 further comprising a polysaccharide.

44. The depot of claim 42 wherein the carrier base material comprises a melting temperature of about 36 degrees C. to about 38 degrees C.

45. The depot of claim 42 wherein the carrier base material comprises a water soluble carrier base.

46. The depot of claim 42 wherein the carrier base material comprises MBK.

47. The depot of claim 42 further comprising a substantially uniform composition.
